(19) United States
(12) Patent Application Publication
(10) Pub. No.: US 2024/0131350 A1
(43) Pub. Date: Apr. 25, 2024

(54) MULTI-POLE RADIO FREQUENCY DEVICE, METHOD FOR CONTROLLING MULTI-POLE RADIO FREQUENCY DEVICE, AND RADIO FREQUENCY THERAPEUTIC APPARATUS

(71) Applicant: SHENZHEN PENINSULA MEDICAL GROUP, Shenzhen (CN)

(72) Inventors: Yanan LI, Shenzhen (CN); Yongsheng LIANG, Shenzhen (CN); Xiaobing LEI, Shenzhen (CN); Yi DING, Shenzhen (CN)

(73) Assignee: SHENZHEN PENINSULA MEDICAL GROUP, Shenzhen (CN)

(21) Appl. No.: 18/400,890

(22) Filed: Dec. 29, 2023

Related U.S. Application Data
(63) Continuation of application No. PCT/CN2023/101523, filed on Jun. 20, 2023.

(30) Foreign Application Priority Data
Sep. 29, 2022 (CN) 202211225134.4

(51) Int. Cl. A61N 1/40

(52) U.S. Cl. CPC A61N 1/40

(57) ABSTRACT

Disclosed are a multi-pole radio frequency (RF) device, a method for controlling multi-pole RF device, and an RF therapeutic apparatus. The multi-pole RF device includes an RF power supply, a return electrode, a therapeutic electrode, a controller, a first switch switching circuit, and/or a second switch switching circuit. The controller controls the third input terminal and the fourth input terminal of the second switch switching circuit to be switchably connected to the third output terminal and the fourth output terminal. In the unipolar mode, the controller controls the first output terminal and the second output terminal to be electrically connected to the therapeutic electrode and the return electrode respectively. In the bipolar mode, the controller controls the first output terminal and the second output terminal to be connected to at least one of the plurality of electrode slices respectively.

### CROSS-REFERENCE TO RELATED APPLICATIONS

This application is a continuation application of International Application No. PCT/CN2023/101523, filed on Jun. 20, 2023, which claims priority to Chinese Patent Application No. 202211225134.4, filed on Sep. 30, 2022. All of the aforementioned applications are incorporated herein by reference in their entireties.

### TECHNICAL FIELD

The present application relates to the technical field of medical apparatuses, and in particular to a multi-pole radio frequency (RF) device, a method for controlling multi-pole RF device, and an RF therapeutic apparatus.

### BACKGROUND

The radio frequency (RF) therapeutic apparatus generates RF energy on human tissues through two different polarized electrode slices. The RF energy stimulates the subcutaneous tissue to allow the polarized water molecules in the tissue to move at a high speed, and a thermal effect is formed to inactivate the protein of the diseased tissue. Then the protein of the diseased tissue will finally fall off due to rejection by the human body, and a new healthy tissue will generate, thereby achieving the therapeutic purpose. However, during treatment of the existing RF therapeutic apparatus, the electrode design of the existing therapeutic handle is fixed, the therapeutic range corresponding to a single electrode mode is limited, and two interfaces of the RF power supply have different heat quantity when outputting RF energy. The difference between the heat quantity will affect the uniformity of the RF energy outputted from both terminals of the RF power supply. In this case, there will be a difference in the RF energy that is respectively outputted by the therapeutic electrode and the return electrode of the therapeutic handle. In this case, the therapeutic range is limited, and the operation is convenient, which affects the therapeutic effect of the RF therapeutic apparatus.

### SUMMARY

The main purpose of the present application is to provide a multi-pole radio frequency (RF) device, aiming to enable the RF electrode to output uniform RF energy, so that the RF energy can produce a better thermal effect in both parallel and vertical directions of the active layer.

In view of the above objectives, the present application provides a multi-pole RF device including:
an RF power supply provided with a first interface and a second interface, an electric polarity of the first interface is opposite to an electric polarity of the second interface;
a return electrode;
a therapeutic electrode provided with a plurality of electrode slices;
a first switch switching circuit provided with a first input terminal electrically connected to the first interface and a second input terminal electrically connected to the second interface, a first output terminal of the first switch switching circuit is electrically connected to the therapeutic electrode, a second output terminal of the first switch switching circuit is switchably and electrically connected to the return electrode or part of the plurality of the electrode slices, and the first output terminal and the second output terminal are respectively connected to the first input terminal and the second input terminal; and/or
a second switch switching circuit, a third input terminal of the second switch switching circuit is connected to the first interface, a fourth input terminal of the second switch switching circuit is connected to the second interface, and the second switch switching circuit is provided with a third output terminal and a fourth output terminal; and
a controller.

In response to that the multi-pole RF device includes the first switch switching circuit, the controller includes a unipolar mode and a bipolar mode.

In the unipolar mode, the controller is configured to control the first output terminal to be electrically connected to the therapeutic electrode, and control the second output terminal to be electrically connected to the return electrode.

In the bipolar mode, the controller is configured to control the first output terminal and the second output terminal to be connected to at least one of the plurality of electrode slices respectively.

In response to that the multi-pole RF device includes the second switch switching circuit, the controller is configured to control the third input terminal to be switchably connected to the third output terminal, and control the fourth input terminal to be switchably connected to the fourth output terminal.

In an embodiment, in response to that the multi-pole RF device includes the second switch switching circuit, controlling, by the controller, to switch at least once between the connection of the third input terminal and the third output terminal, and the connection of the fourth input terminal and the fourth output terminal.

In an embodiment, the controller is configured to control the second switch switching circuit to work, controlling, by the controller, to switch in a first preset period between the connection of the third input terminal and the third output terminal, and the connection of the fourth input terminal and the fourth output terminal.

In an embodiment, in response to that the multi-pole RF device includes the first switch switching circuit, controlling, by the controller, to control the first output terminal and the second output terminal of the first switch switching circuit being switched at least once between the unipolar mode and the bipolar mode.

In an embodiment, the controller is configured to control the first switch switching circuit to work, controlling the first output terminal and the second output terminal of the first switch switching circuit being switched in a second preset period between the unipolar mode and the bipolar mode.

In an embodiment, the first switch switching circuit and the second switch switching circuit are one same switch switching circuit, and the controller is configured to switch between the unipolar mode and the bipolar mode, and switchably connect the therapeutic electrode and/or the return electrode to the first interface and the second interface respectively.

In an embodiment, a contact area of the return electrode is larger than a contact area of the therapeutic electrode.

The present application further provides a method for controlling the multi-pole RF device, applied to the multi-pole RF device as mentioned above. The method for controlling the multi-pole RF device includes:
determining a user input signal;
in response to that the user input signal is the unipolar mode signal, entering the unipolar mode, controlling the first output terminal to be electrically connected to the therapeutic electrode and the second output terminal to be electrically connected to the return electrode, so as to make electric polarities of the plurality of electrode slices of the therapeutic electrode same;
in response to that the user input signal is the bipolar mode signal, entering the bipolar mode, controlling the first output terminal and the second output terminal to be connected to at least one of the electrode slices respectively, so as to make an electric polarity of at least one electrode slice of the therapeutic electrode opposite to electrical polarities of remaining electrode slices; and
in response to that the user input signal is a combined mode signal, controlling the first output terminal and the second output terminal to be switched at least once between the unipolar mode and the bipolar mode.

In an embodiment, the method for controlling the multi-pole RF device further includes:
in the bipolar mode or the unipolar mode, controlling a connection of the third input terminal and the third output terminal being switched at least once by a connection of the fourth input terminal and the fourth output terminal.

In an embodiment, the method for controlling the multi-pole RF device further includes:
in the bipolar mode or the unipolar mode, controlling the connection of the third input terminal the third output terminal being switched within a first preset period by the connection of the fourth input terminal and the fourth output terminal.

In an embodiment, the method for controlling the multi-pole RF device further includes:
in the bipolar mode or the unipolar mode, switching and communicating the third input terminal and the fourth input terminal with the third output terminal and the fourth output terminal,
during one therapeutic process, a heat quantity generated by connecting any one of the plurality of electrode slices of the therapeutic electrodes to the first interface is W1, and a heat quantity generated by connecting any one of the plurality of electrode slices of the therapeutic electrodes to the second interface is W2, W1/W2 is not less than 0.5 and not greater than 2.0.

In an embodiment, the method for controlling the multi-pole RF device further includes:
controlling the first output terminal and the second output terminal to be switched within a second preset period between the unipolar mode and the bipolar mode.

In an embodiment, the method for controlling the multi-pole RF device further includes:
in the combined mode, during one therapeutic process, a heat quantity generated in the unipolar mode is W3, and a heat quantity generated in the bipolar mode is W4, W3/W4 is not less than 0.5 and not greater than 2.0.

In an embodiment, the method for controlling the multi-pole RF device further includes:
obtaining a temperature of the electrode slice of the therapeutic electrode;
determining whether the temperature of the electrode slice exceeds a preset safety temperature threshold;
in response to that the temperature of the electrode slice exceeds the preset safety temperature threshold, controlling the RF power supply to stop outputting RF energy; and
in response to that the temperature of the electrode slice does not exceed the preset safety temperature threshold, controlling the RF power supply to keep outputting effective RF energy.

The present application further provides an RF therapeutic apparatus including the multi-pole RF device as mentioned above.

In the technical solution of the present application, on the one hand, a second switch switching circuit is provided between the RF power supply, the therapeutic electrode and the return electrode. The second switch switching circuit is controlled to coordinate the connection relationship between the therapeutic electrode and the return electrode with the first interface and the second interface of the RF power supply, so that the first interface and the second interface of the RF power supply can be alternately connected to the third input terminal and the fourth input terminal of the second switch switching circuit of the multi-pole RF device. On the other hand, the first switch switching circuit is configured to change the connection relationship between the therapeutic electrode and the return electrode with the first interface and the second interface, so that the multi-pole RF device can be switched between the unipolar mode and the bipolar mode. In this way, the first interface and the second interface of the RF power supply can be continuously switched between the positive electrode and the negative electrode, to uniformly output the RF energy and ensure good thermal effects generated in the parallel and vertical direction, thereby achieving uniform action effect of the energy in the three-dimension. Of course, the second switching circuit and the first switching circuit may be provided together, or only one of the second switching circuit and the first switching circuit may be provided.

### BRIEF DESCRIPTION OF THE DRAWINGS

To illustrate the technical solutions according to embodiments of the present application or the related art more clearly, the accompanying drawings for describing the embodiments or the related art are introduced briefly in the following. Apparently, the accompanying drawings in the following description are only some embodiments of the present application. Those skilled in the art can derive other drawings from the structures of the accompanying drawings without creative efforts.

FIG. 1 is a schematic structural view of a multi-pole radio frequency (RF) device according to an embodiment of the present application.

FIG. 2 is a schematic view showing a distribution of an electrode slice of a therapeutic electrode in FIG. 1 according to an embodiment of the present application.

FIG. 3 is another schematic view showing the distribution of the electrode slice of the therapeutic electrode in FIG. 1 according to an embodiment of the present application.

FIG. 4 is a schematic view showing a distribution of an electrode of the therapeutic electrode in FIG. 1 according to an embodiment of the present application.

FIG. 5 is another schematic structural view of the multi-pole RF device according to an embodiment of the present application.

FIG. 6 is a schematic circuit structure diagram of the multi-pole RF device according to an embodiment of the present application.

FIG. 7 is a schematic flowchart of a method for controlling the multi-pole RF device according to an embodiment of the present application.

FIG. 8 is yet another schematic structural view of the multi-pole RF device according to an embodiment of the present application.

FIG. 9 is yet another schematic structural view of the multi-pole RF device according to an embodiment of the present application.

FIG. 10 is yet another schematic structural view of the multi-pole RF device according to an embodiment of the present application.

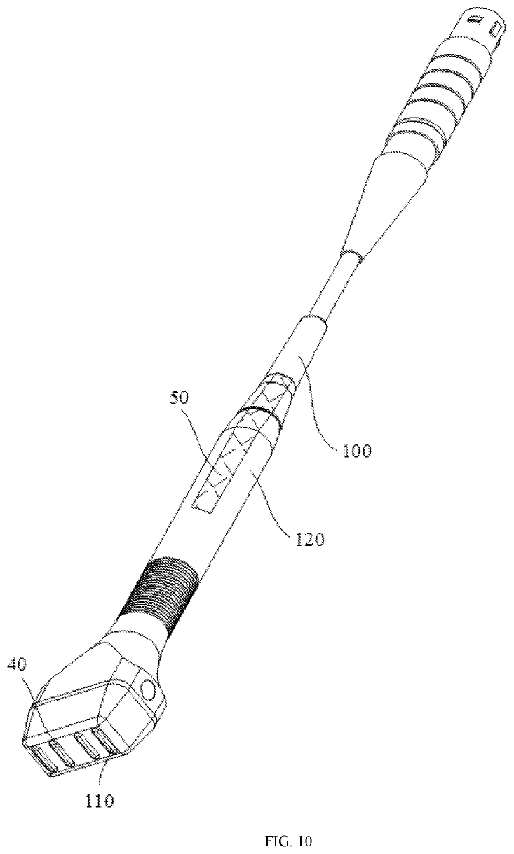

FIG. 11 is yet another schematic structural view of the multi-pole RF device according to an embodiment of the present application.

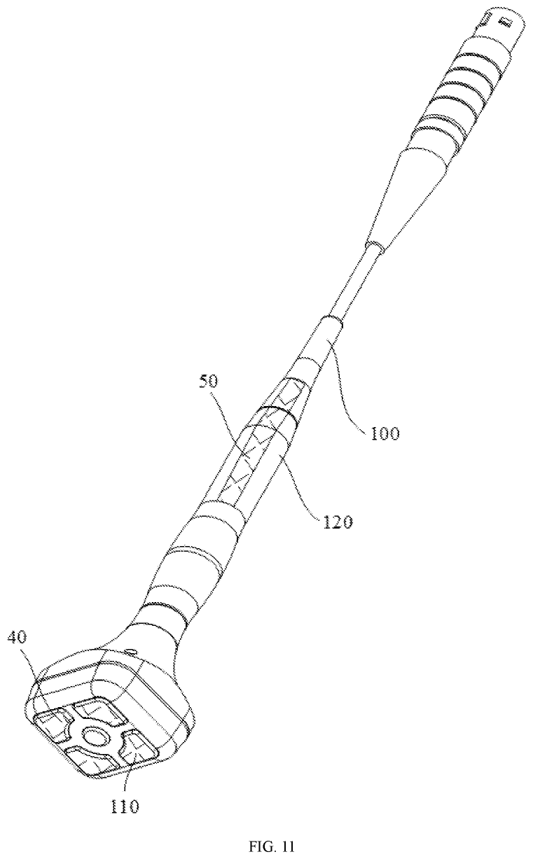

The realization of the objective, functional characteristics, and advantages of the present application are further described with reference to the accompanying drawings.

### DETAILED DESCRIPTION OF THE EMBODIMENTS

The technical solutions of the embodiments of the present application will be described in detail below with reference to the accompanying drawings. It is obvious that the embodiments described are only some rather than all of the embodiments of the present application. All other embodiments obtained by those skilled in the art based on the embodiments of the present application without creative efforts shall fall within the claimed scope of the present application.

It should be noted that all the directional indications (such as up, down, left, right, front, rear . . . ) in the embodiments of the present application are only used to explain the relative positional relationship, movement, or the like of the components in a certain posture (as shown in the drawings). If the specific posture changes, the directional indication will change accordingly.

Besides, the descriptions associated with, e.g., “first” and “second,” in the present application are merely for descriptive purposes, and cannot be understood as indicating or suggesting relative importance or impliedly indicating the number of the indicated technical feature. Therefore, the feature associated with “first” or “second” can expressly or impliedly include at least one such feature. Further, the meaning of “and/or” appearing in the entire text includes three parallel schemes. Taking “A and/or B” as an example, it includes scheme A, or scheme B, or a scheme that satisfies both A and B at the same time. In addition, the technical solutions of the various embodiments can be combined with each other, but the combinations must be based on the realization of those skilled in the art. When the combination of technical solutions is contradictory or cannot be achieved, it should be considered that such a combination of technical solutions does not exist, nor does it fall within the scope of the present application.

In the existing technology, the radio frequency (RF) device generally adopts a single electrode or double electrodes. The action depth of the unipolar RF device is deeper, and the action depth of the bipolar RF device is shallower. On the one hand, users need to replace the unipolar RF device or the bipolar RF device according to different needs during use, resulting a low therapeutic efficiency and poor therapeutic effect. On the other hand, the return electrode of the unipolar RF device needs to be attached to other body regions to achieve a deep therapeutic effect. When the unipolar RF device is used by a single person at home, the operation is too cumbersome, which will reduce the user's desire to use it. In addition, during the therapeutic process, the heat quantity generated by two output terminals of the RF power supply will be different, which may easily cause a heat quantity difference in the RF energy outputted from both terminals of the RF power supply. This heat quantity difference will affect the uniformity of the RF energy outputted from the RF power supply, and uneven RF energy will make the therapeutic effect deviate from the therapeutic expectation, thereby affecting the therapeutic effect of the RF therapeutic apparatus.

The present application provides a multi-pole RF device.

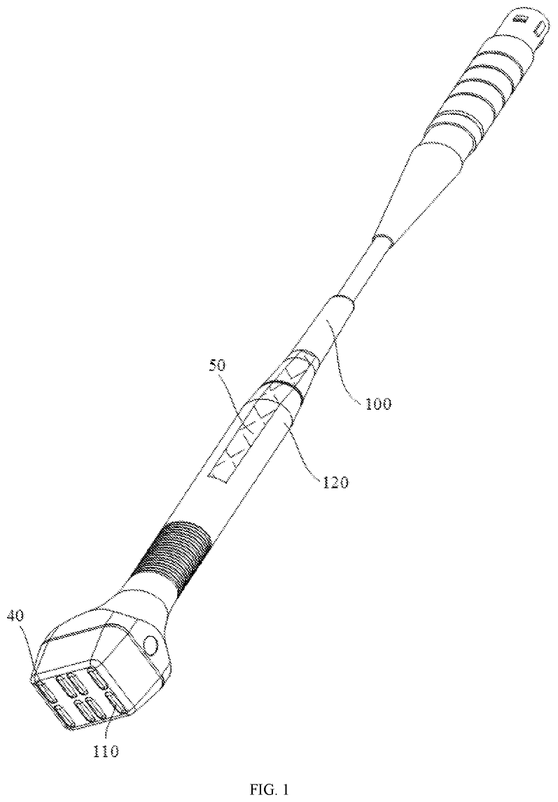

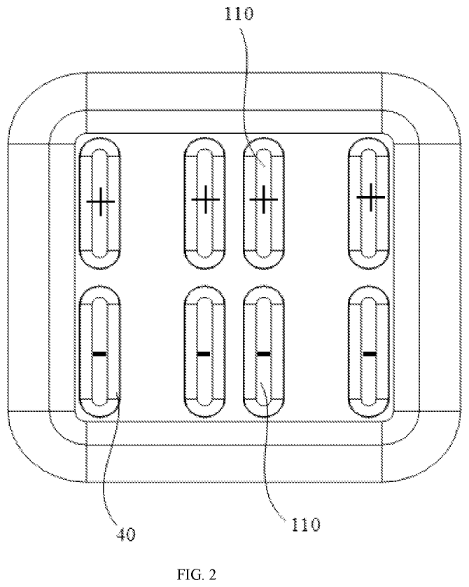

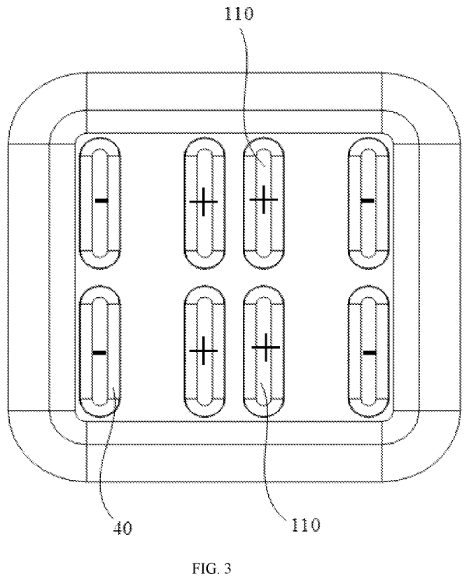

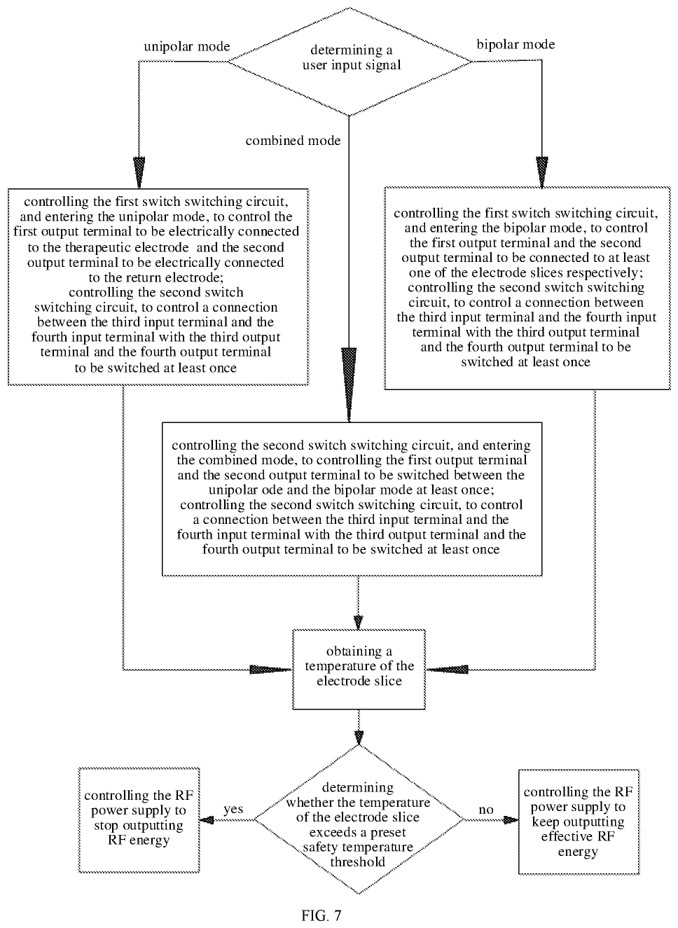

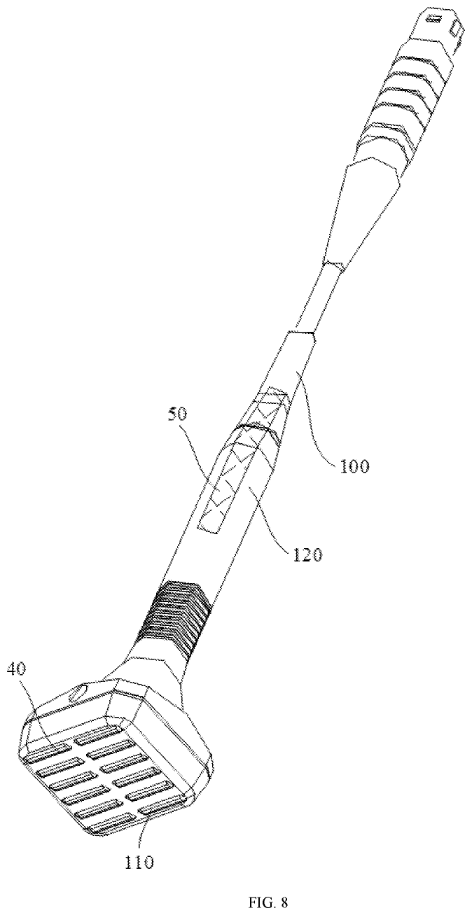

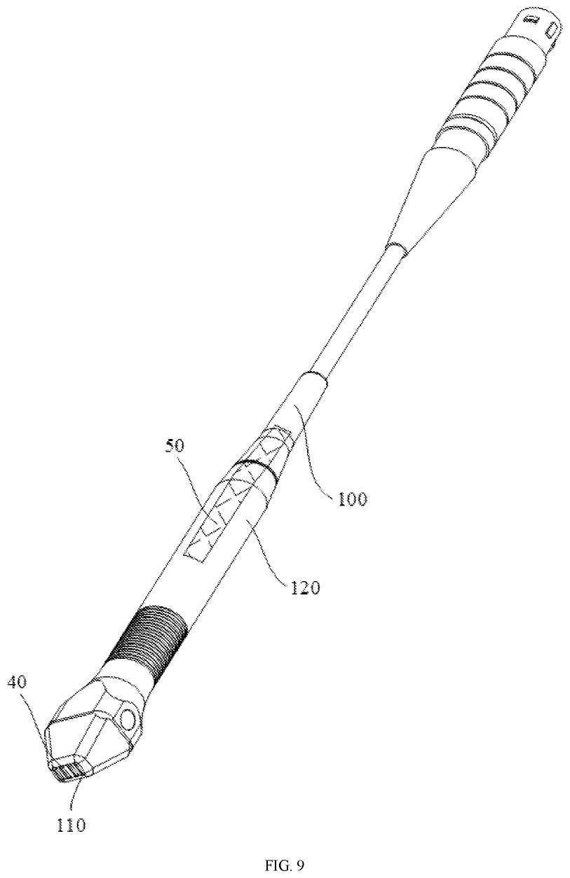

In the embodiments of the present application, as shown in FIG. 1 to FIG. 9, the multi-pole RF device includes an RF power supply 10, a return electrode 50, a therapeutic electrode 40, a first switch switching circuit 30, and/or a second switch switching circuit 20, and a controller 60.

The RF power supply 10 is provided with a first interface and a second interface, and an electric polarity of the first interface is opposite to an electric polarity of the second interface.

The therapeutic electrode 40 includes a plurality of electrode slices 110.

The first switch switching circuit 30 is provided with a first input terminal electrically and a second input terminal electrically connected to the first interface and the second interface respectively. A first output terminal of the first switch switching circuit 30 is electrically connected to the therapeutic electrode 40, and a second output terminal of the first switch switching circuit 30 is switchably and electrically connected to the return electrode 50 or part of the plurality of the electrode slices 110. The first output terminal and the second output terminal are respectively connected to the first input terminal and the second input terminal.

A third input terminal of the second switch switching circuit 20 is connected to the first interface, a fourth input terminal of the second switch switching circuit 20 is connected to the second interface, and the second switch switching circuit 20 is provided with a third output terminal and a fourth output terminal.

When the multi-pole radio frequency device includes the first switch switching circuit 30, the controller 60 includes a unipolar mode and a bipolar mode. In the unipolar mode, the controller 60 controls the first output terminal and the second output terminal to be electrically connected to the therapeutic electrode 40 and the return electrode 50 respectively. In the bipolar mode, the controller 60 controls the first output terminal and the second output terminal to be connected to at least one of the plurality of electrode slices 110 respectively.

When the multi-pole radio frequency device includes the second switch switching circuit 20, the controller 60 controls the third input terminal and the fourth input terminal to be switchably connected to the third output terminal and the fourth output terminal.

In the technical solution of the present application, on the one hand, a second switch switching circuit 20 is provided between the RF power supply 10, the therapeutic electrode 40 and the return electrode 50. The second switch switching circuit 20 is controlled to coordinate the connection relationship between the therapeutic electrode 40 and the return electrode 50 with the first interface and the second interface of the RF power supply 10, so that the first interface and the second interface of the RF power supply 10 can be alternately connected to the third input terminal and the fourth input terminal of the second switch switching circuit 20 of the multi-pole RF device of the present application. On the other hand, the first switch switching circuit 30 is configured to change the connection relationship between the therapeutic electrode 40 and the return electrode 50 with the first interface and the second interface, so that the multi-pole RF device can be switched between the unipolar mode and the bipolar mode. In this way, the first interface and the second interface of the RF power supply 10 can be continuously switched between the positive electrode and the negative electrode, to uniformly output the RF energy and ensure good thermal effects generated in the parallel and vertical direction, thereby achieving uniform action effect of the energy in the three-dimension. Of course, the second switching circuit 20 and the first switching circuit 30 may be provided together, or only one of the second switching circuit 20 and the first switching circuit 30 may be provided.

In the second switch switching circuit 20, the controller 60 can first control the third input terminal to be connected to the third output terminal, and the fourth input terminal to be connected to the fourth output terminal. Then the controller 60 can control the fourth input terminal to be switched to be connected to the third output terminal, and the third input terminal to be switched to be connected to the fourth output terminal, so that when the second switch switching circuit 20 is working, the therapeutic electrode 40 and the return electrode 50 are respectively connected to the first interface or the second interface for the same time, or for a similar time period, to balance the heat quantity of the first interface and the second interface. In this embodiment, when each input terminal and each output terminal in the second switch switching circuit 20 are switched and connected, the first switch switching circuit 30 is controlled by the controller 60 or by a manual operation, to allow all the electrode slices 110 of the therapeutic electrode 40 or part of the electrode slices to be connected to the third output terminal, and allow the return electrode 50 to be connected to the fourth output terminal, so that the therapeutic electrode 40 and the return electrode 50 carry opposite polarities respectively, thereby forming a multi-pole RF device in a unipolar mode. In this embodiment, the first switch switching circuit 30 is additionally controlled by the controller 60 or by a manual operation, to adjust the connection relationship between the plurality of electrode slices 110 of the therapeutic electrode 40 with the first output terminal and the second output terminal respectively, so that at least one of the plurality of the electrode slices 110 is connected to the first output terminal, and the remaining electrode slices 110 or at least one of the remaining electrode slices 110 are connected to the second output terminal. In this way, it can be ensured that the electrical polarity of at least one electrode slice 110 of the therapeutic electrode 40 is opposite to the electrical polarity of at least one of the remaining electrode slices 110, thereby forming a multi-pole RF device in a bipolar mode. Based on the cooperation of the second switch switching circuit 20 and the first switch switching circuit 30 as mentioned above, not only the multi-pole RF device of the present application can be switched between the unipolar mode and the bipolar mode, but also the connection relationship between the first interface and the second interface of the RF power supply 10 with the third input terminal and the fourth input terminal can be constantly converted. In this way, the RF energy of the multi-pole RF device of the present application can be outputted more uniformly on the therapeutic region, to ensure the therapeutic effect.

It should be noted that the electrode slice 110 referred to in this technical solution is only provided at the therapeutic electrode 40. Besides, during one therapeutic process, in the combined mode formed by switching between the unipolar mode and the bipolar mode, the total RF energy outputted in the unipolar mode may be equal to the total RF energy outputted in the bipolar mode. The first input terminal, the second input terminal, the third input terminal, the fourth input terminal, the first output terminal, the second output terminal, the third output terminal, and the fourth output terminal may be multi-branch general terminals or may be the single-branch terminal.

It can be understood that the first switch switching circuit 30 and the second switch switching circuit 20 can be controlled together by the controller. That is, in an embodiment, the first switch switching circuit 30 and the second switch switching circuit 20 can be the same switch switching circuit. The controller 60 is used to switch between the unipolar mode and the bipolar mode, as well as switch between the first interface and the second interface respectively. That is, when in the unipolar mode, the bipolar mode or the combined mode, the controller 60 can further control the mutual switching between the connection ports of the switch switching circuit with the therapeutic electrode 40 and the return electrode 50, to make the heat quantity difference of the first interface and the second interface tend to zero. Or, the switch switching circuit only retains the mutual switching between the first interface and the second interface, to ensure that during one therapeutic process, the total RF energy outputted from the first interface of the RF power supply tends to be equal to the total RF energy outputted from the second interface of the RF power supply, thereby ensuring the therapeutic effect.

In an embodiment, as shown in FIG. 1 to FIG. 5, when the multi-pole RF device includes the second switch switching circuit 20, the controller 60 is configured to control the second switch switching circuit to work 20 to control the connection between the third input terminal and the fourth input terminal with the third output terminal and the fourth output terminal to be switched at least once. It should be noted that during the therapeutic process, if the connection point between the positive electrode and the negative electrode with the RF power supply 10 keeps constant, the first interface and the second interface of the RF power supply 10 are prone to generate a heat quantity difference. The controller 60 controls the second switch switching circuit 20 realizes continuously switching the connection between the third input terminal and the fourth input terminal with the third output terminal and the fourth output terminal respectively, so that the positive electrode and the negative electrode enable the heat generated by the RF power supply 10 to be distributed in the first interface and the second interface evenly, to reduce the heat quantity difference between the first interface and the second interface, thereby ensuring uniform output of the RF energy of the RF power supply 10.

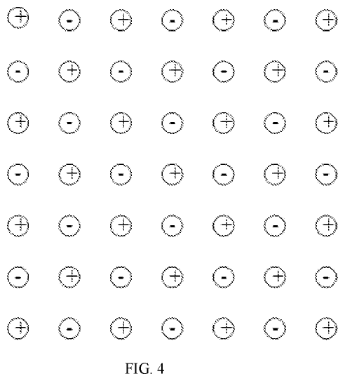

In this embodiment, as shown in FIG. 1 and FIG. 4, the controller 60 is configured to control the second switch switching circuit 20 to work to control the connection between the third input terminal and the fourth input terminal with the third output terminal and the fourth output terminals to be switched within a first preset period. In this way, the controller 60 controls the switching frequency of the second switch switching circuit 20 within the first preset period according to the preset algorithm program. In an embodiment, the duration of the one therapeutic phase ranges from 20 ms to 12 s. The controller 60 can control the switching time of the second switch switching circuit 20 ranges form 0.1 ms to 2 ms, to improve the uniform output of the RF energy and ensure the therapeutic effect. In addition, the controller 60 can also enter a custom mode to set the first preset period according to the user's usage habits.

In an embodiment, as shown in FIG. 1 to FIG. 5. when the multi-pole RF device includes the first switch switching circuit 30, the controller 60 is configured to control the first switch switching circuit 30 to work, to control the first output terminal and the second output terminal to be switched between the unipolar mode and the bipolar mode at least once. In the unipolar mode, the electrode slices 110 on the therapeutic electrode 40 have the same electric polarity, for example, all of the electrode slices 110 are positive electrodes. The return electrode 50 has the same electric polarity, for example, all of the return electrode 50 are negative electrodes. In this case, controlled by the first switch switching circuit 30, the electric polarity of the therapeutic electrode 40 is opposite to the electric polarity of the return electrode 50. Since the human body regions corresponding to the therapeutic electrode 40 and the return electrode 50 are widely spaced, the RF energy outputted from the electrode slices 110 of the therapeutic electrode 40 can act on deeper tissues, to treat the to be treated region in the vertical direction. In the bipolar mode, the electric polarity of at least one electrode slice 110 of the therapeutic electrode 40 is opposite to the electric polarity of the remaining electrode slices 110. The two electrode slices 110 with opposite polarities are provided at the therapeutic electrode 40, so that in the bipolar mode, the path of the RF energy in the human body is shorter, and the RF energy can only act on shallower regions of the human body, to treat the to be treated region in the vertical direction. In this way, the controller 60 controls the first switch switching circuit 30 to realize switching between the unipolar mode and the bipolar mode, so that during one therapeutic process, the RF energy outputted in the unipolar mode is equal to the RF energy outputted in the bipolar mode, which can ensure that the thermal effect of the multi-pole RF device in the present application is distributed in the three-dimension, thereby improving the therapeutic effect.

In this embodiment, as shown in FIG. 4, the controller 60 is configured to control the first switch switching circuit 30 to work, to control the first output terminal and the second output terminal to be switched between the unipolar mode and the bipolar mode within the second preset period. In this way, the user can select the preset switching period according to actual needs, and the user can use the controller 60 to execute the second preset period instruction, to control the working duration and switching duration of the multi-pole RF device in the unipolar mode and the bipolar mode respectively. Or, the user enters the custom mode to select a suitable working period, and saves the customized data to the controller 60, so that the controller 60 will switch between the unipolar mode and the bipolar mode based on the customized data. In this way, the active region of the electrode slice 110 can allow the thermal effect to be distributed in the parallel direction and the vertical direction relative to the region to be treated, thereby forming a better therapeutic effect in three-dimension. It should be noted that, regardless of the system preset period or customized period, during one therapeutic process, the RF energy outputted in the unipolar mode is equal to the RF energy outputted in the bipolar mode. In addition, in other embodiments, it is necessary to manually switch between the unipolar mode and the bipolar mode during use, or to change the working time of the unipolar mode or the bipolar mode at any time according to the usage conditions. In this way, the user can manually control the switch button 130 at the therapeutic handle 100, to output the switching instruction to the controller 60, and the controller 60 switches between the unipolar mode and the bipolar mode.

In an embodiment, the controller 60 may be a microprocessor, such as a microcontroller, a digital signal processor (DSP), or a field programmable gate array (FPGA), and the like, or may be implemented by a dedicated chip for the RF therapeutic apparatus, which will not be limited here. Those skilled in the art can integrate some hardware circuits and preset algorithm programs in the controller 60 and use the controller 60 to run the algorithm program, so that the switching between the unipolar mode and the bipolar mode of the multi-pole RF device of the present application can be automatically realized, and the second switch switching circuit 20 can work. The user can also select the corresponding RF frequency or the working time of the second switch switching circuit 20 in the unipolar mode or the bipolar mode according to the actual needs.

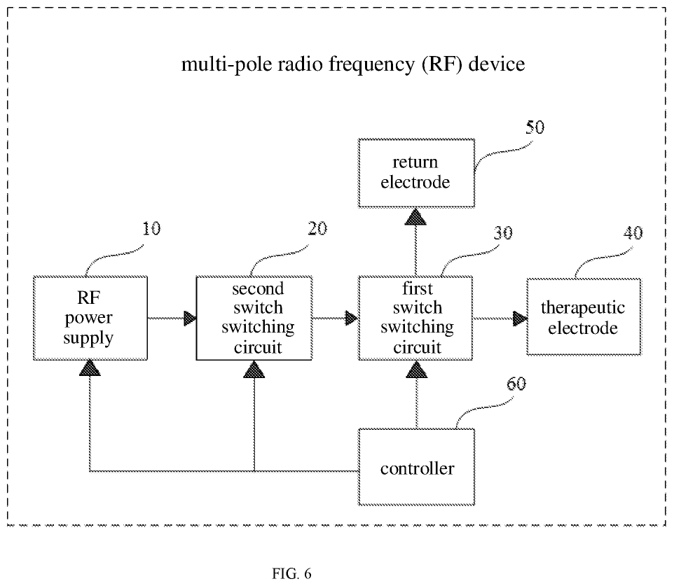

As shown in FIG. 6 to FIG. 9, the plurality of electrode slices 110 of the therapeutic electrode 40 can be distributed in an array, or in a line, or in a circle. In the bipolar mode, the electric polarity of each electrode slice 110 can be the same as or be different from the electric polarity of the adjacent electrode slice 110. For example, the electrode slices 110 are distributed in rows, with positive electrodes and negative electrodes staggered. That is, the electric polarity of the electrode slices 110 in one row is positive, and the electric polarity of the electrode slices 110 in the adjacent row is negative. Or, the electric polarity of each electrode slice 110 is opposite to the electric polarity of its adjacent electrode slice 110. That is, the electrode slices 110 in each row and each column are distributed in a positive polarity and negative polarity staggered manner. Of course, this distribution of the electrode slices 110 can also be applied in the unipolar mode to enhance the RF energy of a single polarity carried by the electrode slices 110 of the therapeutic electrode 40.

Further, in an embodiment, as shown in FIG. 1 and FIG. 6 to FIG. 9, the contact area of the return electrode 50 is larger than the contact area of the therapeutic electrode 40. It should be noted that the size of the contact area between the electrode and the skin relates to the thermal effect of the RF energy. Among the two contact areas formed in the RF circuit, the thermal effect of the RF energy is mainly generated in the region with a smaller contact area. The contact area is the region between the electrode slice 110 or the return electrode 50 with the skin of the region to be treated. In this way, in the unipolar mode, the RF thermal effect is mainly generated by the therapeutic electrode 40 acting on the region to be treated, thereby ensuring the therapeutic effect. Of course, in other embodiments, the contact area of the return electrode 50 may be slightly smaller than the contact area of the electrode slice 110.

In this embodiment, as shown in FIG. 1 and FIG. 6 to FIG. 9, the multi-pole RF device is provided with a holding portion 120, and the return electrode 50 is provided at the holding portion 120. The multi-pole RF device includes a therapeutic handle 100, and the holding portion 120 is provided at the middle of the therapeutic handle 100. When using the holding portion 120, the user generally holds the holding portion 120 with the hand, that is, the user's palm contacts with the return electrode 50. Thus, in the unipolar mode, the electric polarity of the electrode slice 110 of the therapeutic electrode 40 is opposite to the electric polarity of the return electrode 50. In this case, the holding portion 120 can be used as the return electrode 50, and the RF energy outputted by the electrode slice 110 can generate a thermal effect on the region to be treated, thereby achieving a therapeutic effect. Compared with a technical solution that the return electrode 50 is additionally provided to contact other regions of the human body and form a loop, the return electrode 50 is provided at the holding portion 120, which can improve the user's convenience in using the multi-pole RF device and is suitable for individual users at home. Of course, in other embodiments, the return electrode 50 can be provided at another protruding branch of the therapeutic handle 100. When the electrode slice 110 of the therapeutic electrode 40 contacts with the region to be treated, the return electrode 50 can also contact with the human body, to form a loop. Or, in another embodiment, the multi-pole RF device is a large therapeutic device and can be applied in hospitals or beauty institutions. The therapeutic electrode 40 and the return electrode 50 have the communication structure connected to the therapeutic host. The second switch switching circuit 20 and the first switching circuit 30 are provided at the intersection position of the therapeutic electrode 40 and the return electrode 50.

In an embodiment, the RF power supply 10 includes one power supply or a plurality of independent power supplies, and the power supply is connected to the second switching circuit 20. It should be noted that the power supply can be an external power supply or a rechargeable lithium battery, and the power supply can be a continuous output power supply, or a pulse output power supply, or a continuous and pulse output power supply. When only one power supply is adopted, the volume of the multi-pole RF device can be reduced. When a plurality of independent power supplies are adopted, the plurality of power supplies are respectively connected to the third input terminal and the fourth input terminal of the second switch switching circuit 20. The RF power supply 10 also includes a frequency regulator. The frequency regulator can adjust the RF frequency outputted by the RF power supply 10 no matter when the second switch switching circuit 20 is working or when the first switch switching circuit 30 is working, thereby adjusting the therapeutic gear of the multi-pole RF device to achieve better effective therapeutic effect.

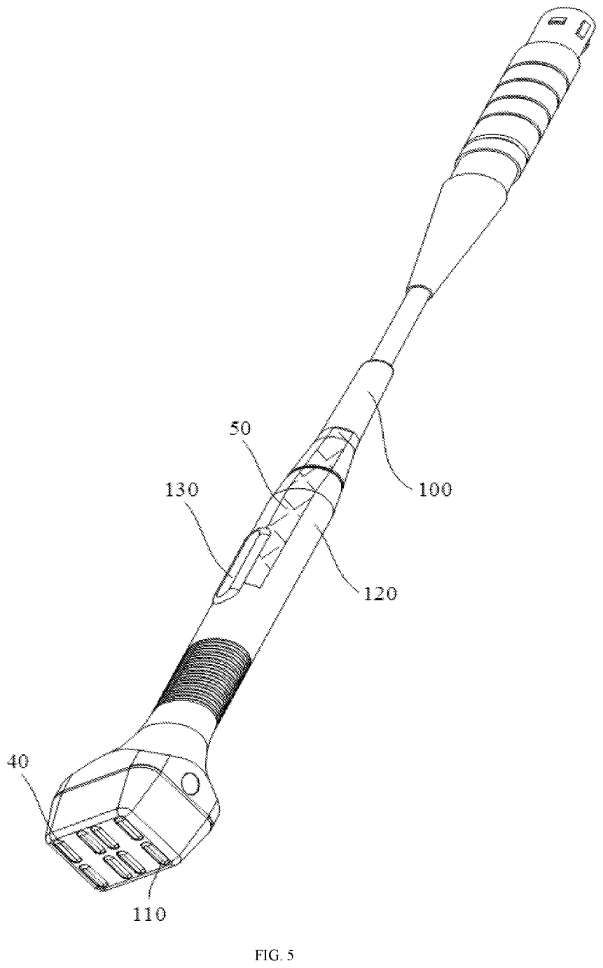

The present application also provides a method for controlling the multi-pole RF device, which is applied to the multi-pole RF device as mentioned above. As shown in FIG. 5, the method for controlling the multi-pole RF device includes:
determining a user input signal;
when the signal is the unipolar mode signal, entering the unipolar mode, controlling the first output terminal and the second output terminal to be electrically connected to the therapeutic electrode 40 and the return electrode 50 respectively, to make electric polarities of the plurality of electrode slices 110 of the therapeutic electrode same;
when the signal is the bipolar mode signal, entering the bipolar mode, controlling the first output terminal and the second output terminal to be connected to at least one of the electrode slices 110 respectively, to make an electric polarity of at least one electrode slice 110 of the therapeutic electrode 40 opposite to an electrical polarity of a remaining electrode slice 110;
when the signal is in a combined mode, controlling the first output terminal and the second output terminal to be switched between the unipolar mode and the bipolar mode at least once.

The user can determine whether to use the unipolar mode, or the bipolar mode, or use both the unipolar mode or the bipolar mode according to the position of the region to be treated. According to user's determination result, an execution signal of the corresponding result is sent to the controller 60. The method for controlling the multi-pole RF device is based on the user's determination of the relative skin depth of the region to be treated, which delivers RF energy acting on target tissues in different depths ranges. Since the return electrode 50 is disposed on the holding portion 120 of the multi-pole RF device, the unipolar mode has a wider distance between the positive electrode and the negative electrode, the resistive heating of the tissue by RF energy is wider and deeper, therefore the unipolar mode can deliver RF energy into a deeper tissue compared with the bipolar mode.

The RF energy is impossible to control precisely within the heated tissue according to the spatial and depth, because the resistive heating of tissues by RF energy occurs along the entire path of electrical conduction through tissues. Therefore, in the present application, the depth of the target tissue varies in three levels, relatively deep, moderate and relatively shallow, initiating three modes for users to select. The controller may control the delivery of RF energy at a suitably depth, distribution and timing to achieve the desired therapeutic effect of thermal injury to treat the target tissue, specifically, spatial control of a treatment depth can be suitably adjusted in three ranges. In one exemplary embodiment, the target tissue includes a specific region of the SMAS region underlying the epidermis, in addition, the SMAS region varies in depth and thickness at different locations of the face, e.g., between 0.5 mm to 5 mm or more. Important structures such as nerves, parotid gland, arteries and veins are present over, under or near the SMAS region. Tightening of the SMAS in the aforementioned locations need to delivery RF energy at different depths.

In this embodiment, when the depth of the region to be treated is relatively deep, the user inputs a unipolar mode signal, and the controller 60 controls the first switch switching circuit 30 to be switched to the unipolar mode. In the unipolar mode, the first switch switching circuit 30 is controlled by the controller 60 to connect the therapeutic electrode 40 with the third output terminal, and connect the return electrode 50 with the fourth output terminal, so that the electric polarity of the therapeutic electrode 40 is opposite to the electric polarity of and the return electrode 50. Since the path formed by the therapeutic electrode 40 and the return electrode 50 on the human body is relatively long, and the therapeutic electrode 40 can produce a thermal effect on the deeper region to be treated, so that the RF energy can penetrate deeply into the skin.

When the depth of the region to be treated is relatively shallow, the user inputs a bipolar mode signal, and the controller 60 controls the first switch switching circuit 30 to be switched to the bipolar mode. In the bipolar mode, a part of the electrode slices of the therapeutic electrode 40 are connected to the third output terminals, and another part of the electrode slices are connected to the fourth output terminal, so that the electric polarity of at least one electrode slice 110 on the therapeutic electrode 40 is opposite to the electric polarity of the remaining electrode slices 110. Since the path formed by the electrode slice 110 of the therapeutic electrode 40 with opposite electric polarities is relative short on the human body, the electrode slice 110 with a smaller contact area among the two electrode slices 110 that include opposite electric polarities can produce a thermal effect on the shallower region to be treated.

When the depth of the region to be treated relative to the skin is moderate and suitable for switching between the unipolar mode and the bipolar mode, that is, the multi-pole RF device is in the combined mode, the user inputs a combined mode signal, and the controller 60 controls the first switch switching circuit 30 to be switched between the unipolar mode and the bipolar mode at least once. Effective therapeutic effect cannot be achieved by relying only on a single mode. The working mode of the multi-pole RF device of the present application can be the unipolar mode or the bipolar mode when the multi-pole RF device initially starts up. After a period of time, the unipolar mode is switched to the bipolar mode, or the bipolar mode is switched to the unipolar mode. The working mode is switched at least once during the entire therapeutic phase. Since the action region in unipolar mode is different from the action region in bipolar mode, in order to expand the action region and improve the therapeutic effect, a working mode of switching between the unipolar mode and the bipolar mode is adopted in this embodiment, so that the expected therapeutic effect can be achieved on the regions to be treated in both parallel and vertical directions relative to the skin. It should be noted that in this embodiment, during one therapeutic process of the combined mode, the total RF energy outputted in the unipolar mode is equal to the total RF energy outputted in the bipolar mode. That is, during the working time respectively corresponding to the unipolar mode and the bipolar mode, the total RF energy outputted in the unipolar mode is equal to the total RF energy outputted in the bipolar mode.

In an embodiment, in the bipolar mode or the unipolar mode, the connection between the third input terminal and the fourth input terminal with the third output terminal and the fourth output terminal are controlled to be switched at least once. It can be understood that the multi-pole RF device is in the unipolar mode or the bipolar mode means that the multi-pole RF device is only in the unipolar mode, only in the bipolar mode, and only in the combined mode. Controlled by the controller 60, the second switch switching circuit 20 can allow the third input terminal and the fourth input terminal to be respectively connected to the third output terminal and the fourth output terminal, or allow the third input terminal and the fourth input terminal to be connected to the fourth output terminal and the third output terminal respectively, or allow the connection between the third input terminal and the fourth input terminal with the third output terminal and the fourth output terminal to be switched at least once, to ensure that in the unipolar mode or bipolar mode, the two interfaces of the RF power supply 10 output relatively uniform RF energy, thereby achieving a better therapeutic effect.

Further, in an embodiment, the method for controlling the multi-pole RF device further includes in the bipolar mode or the unipolar mode, controlling the connection between the third input terminal and the fourth input terminal with the third output terminal and the fourth output terminal to be switched within the first preset period. During treatment, in order to ensure that the heat quantity difference between the first interface and the second interface of the RF power supply 10 can tend to zero, that is, when the second switch switching circuit 20 is working, the therapeutic electrode 40 and the return electrode 50 are respectively connected to the first interface or the second interface for the same time, or for a similar time period. In this way, the controller 60 controls the second switch switching circuit 20 to work within the first preset period. In the unipolar mode or the bipolar mode, controlled by the controller 60, the second switch switching circuit 20 can switch the connections between the third input terminal and the fourth input terminal with the third output terminal and the fourth output terminal within the first period, so that the first interface and the second interface of the RF power supply 10 can output RF energy relatively evenly, thereby achieving the expected therapeutic effect.

Further, in this embodiment, in the bipolar mode or the unipolar mode, switching between the third input terminal and the fourth input terminal with the third output terminal and the fourth output terminal is communicated. During one therapeutic process, the heat quantity generated by connecting any one of the electrode slices 110 of the therapeutic electrode 40 to the first interface is W1, and the heat quantity generated by connecting any one of the electrode slices 110 of the therapeutic electrode 40 to the second interface is W2. W1/W2 is not less than 0.5 and not greater than 2.0. It should be noted that W1 represents the accumulated heat quantity generated by any electrode slice 110 of the therapeutic electrode 40 corresponding to the first interface, and W2 represents the accumulated heat quantity generated by any electrode slice 110 of the therapeutic electrode 40 corresponding to the second interface. It can be understood that, regardless of the unipolar mode or the bipolar mode, during one therapeutic time, W1 refers the product of the working time of the electrode slice 110 corresponding to the first interface and the corresponding power, and W2 refers the product of the working time of the electrode slice 110 corresponding to the second interface and the corresponding power. More specifically, W1 refers the RF energy outputted on the electrode slice 110 by the first interface, and W2 refers the RF energy outputted on the electrode slice 110 by the second interface. That is, during one therapeutic process, the ratio of the RF energy generated by the first interface and the second interface on the therapeutic electrode 40 respectively ranges from 0.5 to 2.0. In a specific embodiment, W1/W2 can be 0.5, 1, or 2.

In an embodiment, the method for controlling the multi-pole RF device further includes controlling the first output terminal and the second output terminal to be switched between the unipolar mode and the bipolar mode within a second preset period. On the Basis that the relative depth of the user's skin to be treated is moderate, the controller 60 controls the first switch circuit 30 to work within a second preset period during the therapeutic process. The multi-pole RF device of the present application initially starts, and the working mode may be one of the unipolar mode or the bipolar mode. Then the working mode will be switched to the other mode within the second preset period. The working mode during the entire therapeutic phase is the combined mode, and is switched within the second preset period, but it is necessary to ensure that during one therapeutic process, when the multi-pole RF device is in the combined mode, the total RF energy outputted in the unipolar mode is equal to the total RF energy outputted in the combined mode. Since the action region in the unipolar mode is different from the action region in the bipolar mode, the working mode of switching between the unipolar mode and the bipolar mode is adopted in this embodiment, so that the expected therapeutic effect can be achieved on the region to be treated in both parallel and vertical directions relative to the skin. Of course, the user can also use the controller 60 to treat in the unipolar mode and the bipolar mode at the customized period according to the adjustments and changes of the actual region to be treated, or the user can manually input instructions to the controller 60 through the switch button 130, and then the controller 60 switches between the unipolar mode and the bipolar mode.

Further, in this embodiment, when the multi-pole RF device is in the combined mode, during one therapeutic process, the heat quantity generated in the unipolar mode is W3, and the heat quantity generated in the bipolar mode is W4. W3/W4 is not less than 0.5 and not greater than 2.0. It should be noted that W3 represents the accumulated heat quantity generated in the unipolar mode during one therapeutic process in the combined mode, and W4 represents the accumulated heat quantity generated in the bipolar mode during one therapeutic process in the combined mode. It can be understood that, in the combined mode, W3 refers the product of the working time in the unipolar mode during one therapeutic process and the corresponding power, and W4 refers the product of the working time in the bipolar mode during one therapeutic process and the corresponding power, namely the heat quantity generated by the region to be treated at the deep layer in the unipolar mode and the heat quantity generated by the region to be treated at the shallow layer in the bipolar mode. In this way, the quantitative ratio of the respective thermal effects in the parallel and vertical directions of the region to be treated relative to the skin ranges from 0.5 to 2. The multi-pole RF device of the present application can produce a uniform thermal effect in the region to be treated along three-dimension direction, thereby achieving better therapeutic effects. In an embodiment, W3/W4 may be 0.5, 1, or 2.

In order to better control the output energy of the multi-pole RF device, in an embodiment, the method for controlling the multi-pole RF device further includes:
obtaining a temperature of the electrode slice 110 of the therapeutic electrode 40;
determining whether the temperature of the electrode slice 110 exceeds a preset safety temperature threshold;
when the temperature of the electrode slice 110 exceeds the preset safety temperature threshold, controlling the radio frequency power supply 10 to stop outputting radio frequency energy; and
when the temperature of the electrode slice 110 does not exceed the preset safety temperature threshold, controlling the radio frequency power supply 10 to keep outputting effective radio frequency energy.

In this embodiment, a temperature sensor can be provided at the therapeutic electrode 40 to detect the temperature on any electrode slice 110. As long as the multi-pole RF device of the present application is working, the temperature sensor detects the temperature of the electrode slice 110, regardless of whether the multi-pole RF device is in the unipolar mode, or the bipolar mode, or the combined mode, or the second switch switching circuit works together or alone.

It should be noted that the effective therapeutic temperature of the RF therapeutic apparatus ranges from 38° C. and 48° C. Within this temperature range, the electrode slice 110 on the therapeutic electrode 40 can effectively produce a thermal effect on the therapeutic region, thereby achieving the treatment purpose. In an embodiment, the safety temperature threshold can be set to 45° C., 48° C. or 49° C. according to different gears. In this way, according to the temperature on the electrode slice 110 detected by the temperature sensor, this temperature is compared with the safety temperature threshold.

When the temperature is greater than the safety temperature threshold, the controller 60 controls the RF power supply 10 to stop outputting RF energy. When the temperature is less than or equal to the safety temperature threshold, the controller 60 controls the RF power supply 10 to keep outputting effective energy.

The present application also provides an RF therapeutic apparatus. The therapeutic apparatus includes a multi-pole RF device. The specific structure of the multi-pole RF device can refer to the embodiments as mentioned above. Since this RF therapeutic apparatus adopts all the technical solutions of all the embodiments as mentioned above, it has at least all the beneficial effects brought by the technical solutions of the above embodiments, which will not be repeated here.

The above are only some embodiments of the present application, and do not limit the scope of the present application thereto. Under the concept of this application, any equivalent structural transformation made according to the description and drawings of the present application, or direct/indirect application in other related technical fields shall fall within the claimed scope of the present application.

What is claimed is:

1. A multi-pole radio frequency (RF) device, comprising:
an RF power supply provided with a first interface and a second interface, wherein an electric polarity of the first interface is opposite to an electric polarity of the second interface;
a return electrode;
a therapeutic electrode provided with a plurality of electrode slices;
a first switch switching circuit provided with a first input terminal electrically connected to the first interface and a second input terminal electrically connected to the second interface, wherein a first output terminal of the first switch switching circuit is electrically connected to the therapeutic electrode, a second output terminal of the first switch switching circuit is switchably and electrically connected to the return electrode or part of the plurality of the electrode slices, and the first output terminal and the second output terminal are respectively connected to the first input terminal and the second input terminal; and/or
a second switch switching circuit, wherein a third input terminal of the second switch switching circuit is connected to the first interface, a fourth input terminal of the second switch switching circuit is connected to the second interface, and the second switch switching circuit is provided with a third output terminal and a fourth output terminal; and
a controller;
wherein in response to that the multi-pole RF device comprises the first switch switching circuit, the controller comprises a unipolar mode and a bipolar mode;
in the unipolar mode, the controller is configured to control the first output terminal to be electrically connected to the therapeutic electrode, and control the second output terminal to be electrically connected to the return electrode;
in the bipolar mode, the controller is configured to control the first output terminal and the second output terminal to be connected to at least one of the plurality of electrode slices respectively; and
in response to that the multi-pole RF device comprises the second switch switching circuit, the controller is configured to control the third input terminal to be switchably connected to the third output terminal, and control the fourth input terminal to be switchably connected to the fourth output terminal.

2. The multi-pole RF device according to claim 1, wherein in response to that the multi-pole RF device comprises the second switch switching circuit, controlling, by the controller, to switch at least once between the connection of the third input terminal and the third output terminal, and the connection of the fourth input terminal and the fourth output terminal.

3. The multi-pole RF device according to claim 2, wherein the controller is configured to control the second switch switching circuit to work, controlling, by the controller, to switch in a first preset period between the connection of the third input terminal and the third output terminal, and the connection of the fourth input terminal and the fourth output terminal.

4. The multi-pole RF device according to claim 1, wherein in response to that the multi-pole RF device comprises the first switch switching circuit, controlling, by the controller, the first output terminal and the second output terminal of the first switch switching circuit to be switched at least once between the unipolar mode and the bipolar mode.

5. The multi-pole RF device according to claim 4, wherein the controller is configured to control the first switch switching circuit to work, controlling the first output terminal and the second output terminal of the first switch switching circuit to be switched in a second preset period between the unipolar mode and the bipolar mode.

6. The multi-pole RF device according to claim 1, wherein the first switch switching circuit and the second switch switching circuit are one same switch switching circuit, and the controller is configured to switch between the unipolar mode and the bipolar mode, and switchably connect the therapeutic electrode and/or the return electrode to the first interface and the second interface respectively.

7. The multi-pole RF device according to claim 1, wherein a contact area of the return electrode is larger than a contact area of the therapeutic electrode.

8. A method for controlling the multi-pole RF device, applied to the multi-pole RF device according to claim 1, comprising:
determining a user input signal according to different depths of regions under a skin surface;
wherein in response to that the user input signal is the unipolar mode signal for a relatively shallow region to be treated, entering the unipolar mode, controlling the first output terminal to be electrically connected to the therapeutic electrode and the second output terminal to be electrically connected to the return electrode, so as to make electric polarities of the plurality of electrode slices of the therapeutic electrode same;
wherein in response to that the user input signal is the bipolar mode signal for a relatively deep region to be treated, entering the bipolar mode, controlling the first output terminal and the second output terminal to be connected to at least one of the electrode slices respectively, so as to make an electric polarity of at least one electrode slice of the therapeutic electrode opposite to electrical polarities of remaining electrode slices; and
wherein in response to that the user input signal is a combined mode signal for a moderate region to be treated, controlling the first output terminal and the second output terminal to be switched at least once between the unipolar mode and the bipolar mode.

9. The method for controlling the multi-pole RF device according to claim 8, further comprising:
in the bipolar mode or the unipolar mode, controlling a connection of the third input terminal and the third output terminal being switched at least once by a connection of the fourth input terminal and the fourth output terminal.

10. The method for controlling the multi-pole RF device according to claim 9, further comprising:
in the bipolar mode or the unipolar mode, controlling the connection of the third input terminal the third output terminal being switched within a first preset period by the connection of the fourth input terminal and the fourth output terminal.

11. The method for controlling the multi-pole RF device according to claim 10, further comprising:
in the bipolar mode or the unipolar mode, switching and communicating the third input terminal and the fourth input terminal with the third output terminal and the fourth output terminal,
wherein during one therapeutic process, a heat quantity generated by connecting any one of the plurality of electrode slices of the therapeutic electrodes to the first interface is W1, and a heat quantity generated by connecting any one of the plurality of electrode slices of the therapeutic electrodes to the second interface is W2, wherein W1/W2 is not less than 0.5 and not greater than 2.0.

12. The method for controlling the multi-pole RF device according to claim 8, further comprising:
controlling the first output terminal and the second output terminal to be switched within a second preset period between the unipolar mode and the bipolar mode.

13. The method for controlling the multi-pole RF device according to claim 12, further comprising:
in the combined mode, during one therapeutic process, a heat quantity generated in the unipolar mode is W3, and a heat quantity generated in the bipolar mode is W4, wherein W3/W4 is not less than 0.5 and not greater than 2.0.

14. The method for controlling the multi-pole RF device according to claim 8, further comprising:
obtaining a temperature of the electrode slice of the therapeutic electrode;
determining whether the temperature of the electrode slice exceeds a preset safety temperature threshold;
wherein in response to that the temperature of the electrode slice exceeds the preset safety temperature threshold, controlling the RF power supply to stop outputting RF energy; and
wherein in response to that the temperature of the electrode slice does not exceed the preset safety temperature threshold, controlling the RF power supply to keep outputting effective RF energy.

15. An RF therapeutic apparatus comprising a multi-pole RF device, wherein the multi-pole RF device comprises:
an RF power supply provided with a first interface and a second interface, wherein an electric polarity of the first interface is opposite to an electric polarity of the second interface;
a return electrode;
a therapeutic electrode provided with a plurality of electrode slices;
a first switch switching circuit provided with a first input terminal electrically connected to the first interface and a second input terminal electrically connected to the second interface, wherein a first output terminal of the first switch switching circuit is electrically connected to the therapeutic electrode, a second output terminal of the first switch switching circuit is switchably and electrically connected to the return electrode or part of the plurality of the electrode slices, and the first output terminal and the second output terminal are respectively connected to the first input terminal and the second input terminal; and/or
a second switch switching circuit, wherein a third input terminal of the second switch switching circuit is connected to the first interface, a fourth input terminal of the second switch switching circuit is connected to the second interface, and the second switch switching circuit is provided with a third output terminal and a fourth output terminal; and
a controller;
wherein in response to that the multi-pole RF device comprises the first switch switching circuit, the controller comprises a unipolar mode and a bipolar mode;
in the unipolar mode, the controller is configured to control the first output terminal to be electrically connected to the therapeutic electrode, and control the second output terminal to be electrically connected to the return electrode;
in the bipolar mode, the controller is configured to control the first output terminal and the second output terminal to be connected to at least one of the plurality of electrode slices respectively; and
in response to that the multi-pole RF device comprises the second switch switching circuit, the controller is configured to control the third input terminal to be switchably connected to the third output terminal, and control the fourth input terminal to be switchably connected to the fourth output terminal.
